SEQUENCE LISTING (7 sequences)

SEQ ID NO: 1 - DNA, 34 bp, synthetic construct
  source 1..34: mol_type = other DNA
ttttttttttagtgagctctgaagtcttagaact

SEQ ID NO: 2 - DNA, 24 bp, synthetic construct
  source 1..24: mol_type = other DNA
agttctaagacttcagagctcact

SEQ ID NO: 3 - DNA, 28 bp, synthetic construct
  source 1..28: mol_type = other DNA
ttttttttttcatacggttatagagtag

SEQ ID NO: 4 - DNA, 18 bp, synthetic construct
  source 1..18: mol_type = other DNA
ctactctataaccgtatg

SEQ ID NO: 5 - DNA, 24 bp, synthetic construct
  source 1..24: mol_type = other DNA
cgcatctgtgcggtatttcacttt

SEQ ID NO: 6 - DNA, 21 bp, synthetic construct
  source 1..21: mol_type = other DNA
cgcatctgtgcggtatttcac

SEQ ID NO: 7 - DNA, 21 bp, synthetic construct
  source 1..21: mol_type = other DNA
gtgaaataccgcacagatgcg

(19) United States
(12) Patent Application Publication
(10) Pub. No.: US 2026/0098863 A1
(43) Pub. Date: Apr. 9, 2026

(54) Tension-Activated Cell Tagging (TaCT)

(71) Applicant: Emory University, Atlanta, GA (US)

(72) Inventors: Rong Ma, Atlanta, GA (US); Khalid Salaita, Atlanta, GA (US)

(21) Appl. No.: 19/114,258

(22) PCT Filed: Sep. 21, 2023
(86) PCT No.: PCT/US2023/074757

§ 371 Date: Mar. 21, 2025

Related U.S. Application Data
(60) Provisional application No. 63/408,530, filed on Sep. 21, 2022.

(51) Int. Cl. G01N 33/58; G01N 15/10; G01N 15/149; G01N 33/50; G01N 33/68

(52) U.S. Cl. CPC G01N 33/582; G01N 15/149; G01N 33/5005; G01N 33/6803; G01N 2015/1006

(57) ABSTRACT

This disclosure contemplates tagging cells or vesicles with agents using a tension activated device. In certain embodiments, a solid surface is conjugated to a double stranded nucleic acid complex wherein a first strand is conjugated to the surface containing a ligand, and a second strand is conjugated to a lipophilic agent. In certain embodiments, the first and/or second strand is conjugated to a label, e.g., fluorescent dye. In certain embodiments, a cell or vesicle comprising a receptor that binds to the ligand provides tension on the double stranded complex to denature the strands resulting the cell membrane associating with or incorporating the lipid conjugated strand.

### CROSS-REFERENCE TO RELATED APPLICATIONS

This application claims the benefit of U.S. Provisional Application No. 63/408,530 filed Sep. 21, 2022. The entirety of this application is hereby incorporated by reference for all purposes.

### STATEMENT REGARDING FEDERALLY SPONSORED RESEARCH OR DEVELOPMENT

This invention was made with government support under GM124472 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22182PCT.xml. The XML file is 7 KB, was created on Sep. 20, 2023, and is being submitted electronically via the USPTO patent electronic filing system.

### BACKGROUND

One can use antibodies and complementary nucleic acids to fluorescently label cells followed by analysis and sorting using flow cytometry and Fluorescence-activated Cell Sorting (FACS). Flow cytometry is typically used for characterizing single-cell molecular expression levels, i.e., the biochemical profile of single cells. However, there is a need to identify improvements.

Wang et al. report a tension gauge tether (TGT) approach in which a ligand is immobilized to a surface through a rupturable tether before receptor engagement. Science, 2013, 40 (6135): 991-4.

Liu et al. report DNA-based nanoparticle tension sensors reveal that T-cell receptors transmit defined pN forces to their antigens for enhanced fidelity. PNAS, 2016, 113:5610-5615.

Zhao et al. report lipid-oligonucleotide conjugates for simple and efficient cell membrane engineering and bioanalysis. Current Opinion in Biomedical Engineering, 2020, 13:76-83.

Hu et al. report DNA-based microparticle tension sensors (mTS) for measuring cell mechanics in non-planar geometries and for high-throughput quantification. Angew Chem Int Ed, 2021, 60, 18044-18050.

Baek et al. report molecular tension probes to quantify cell-generated mechanical forces. Mol Cells, 2022, 45 (1): 26-32.

Ma et al. report the magnitude of LFA-1/ICAM-1 forces fine-tune TCR-triggered T cell activation. Sci Adv, 2022, 8, eabg4485.

Zhang et al. report de novo labeling and trafficking of individual lipid species in live cells. Mol Metab, 2022, 61:101511.

References cited herein are not an admission of prior art.

### SUMMARY

This disclosure contemplates tagging cells or vesicles with agents using a tension activated device. In certain embodiments, a solid surface is conjugated to a double stranded nucleic acid complex wherein a first strand is conjugated to the surface containing a ligand, e.g., on the opposite end, and a second strand is conjugated to a lipophilic agent. In certain embodiments, the first and/or second strand is conjugated to a label, e.g., fluorescent dye. In certain embodiments, a cell or vesicle comprising a receptor that binds to the ligand provides tension on the double stranded complex to denature the strands resulting the cell membrane associating with or incorporating the lipid conjugated strand.

In certain embodiments, this disclosure relates to methods of fluorescently labeling a cell comprising contacting 1) a device comprising: a) a surface, b) a first strand of a nucleobase polymer linked to an area of the surface, wherein the first strand is conjugated to a ligand, and c) a second strand of a nucleobase polymer hybridized with the first strand forming a duplex, wherein the second strand is conjugated to a lipophilic agent, wherein the second strand is conjugated to a fluorescent dye; and 2) a cell comprising a receptor to the ligand and a lipid membrane applying receptor-mediated tension to the device whereby the lipophilic agent of the second strand incorporates to the lipid membrane of the cell and/or associates with the cell membrane thereby fluorescently labeling the cell.

In certain embodiments, the first strand is conjugated to a fluorescent dye, and the second strand is conjugated to a fluorescent dye.

In certain embodiments, methods further comprise identifying the receptor binding to the ligand by detecting fluorescence of the fluorescent dye conjugated the second strand on the cell and/or detecting the fluorescence of the fluorescent dye conjugated the first strand on the surface.

In certain embodiments, the first strand is conjugated to a fluorescent dye, and the fluorescent dye is a fluorescent donor, and the second strand fluorescent dye is a fluorescent quencher. In certain embodiments, the method further comprises detecting fluorescence of the fluorescent quencher on the cell.

In certain embodiments, the first strand is conjugated to a fluorescent dye, and the fluorescent dye is a fluorescent quencher, and the second strand fluorescent dye is a fluorescent donor. In certain embodiments, the method further comprises identifying the receptor binding to the ligand by detecting fluoresce on the surface and/or on the cell.

In certain embodiments, methods further comprise purifying the fluorescently labeled cell by fluorescent activated cell sorting and optionally sequencing genetic material of the cell, e.g., DNA or mRNA in the purified cell.

In certain embodiments, the nucleobase polymer is DNA or RNA.

In certain embodiments, the cell or vesicle is a platelet. In certain embodiments, the ligand is an RGD peptide.

In certain embodiments, the lipophilic agent is a sterol. In certain embodiments, the lipophilic agent is a cholesterol.

In certain embodiments, this disclosure relates to methods of fluorescently identifying a cell or vesicle for receptor ligand binding comprising contacting 1) a device comprising: a) a surface, b) a first strand of a nucleobase polymer linked to an area of the surface, wherein the first strand is conjugated to a ligand and optionally conjugated to a fluorescent dye or other label and c) a second strand of a nucleobase polymer hybridized with the first strand forming a duplex, wherein the second strand is conjugated to a lipophilic agent, wherein the second strand is optionally conjugated to a fluorescent dye or other label; and 2) a cell or vesicle comprising a receptor to the ligand and a lipid membrane applying receptor-mediated tension to the device at the receptor opposite the ligand after the contacting step whereby the receptor binds the ligand providing receptor-mediated tension, e.g., due to stretching the duplex and the receptor-mediated tension exerts forces exceeding a total force needed to denature the duplex by peeling, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell or associates with the cell membrane providing a tagged cell or vesicle.

In certain embodiments, the label is a fluorescent dye. In certain embodiments, the first strand is conjugated to a fluorescent donor and the second strand is conjugated to a fluorescent quencher. In certain embodiments, the first strand is conjugated to a fluorescent quencher and the second strand is conjugated to a fluorescent donor.

In certain embodiments, methods further comprise identifying the receptor binding and/or quantifying the tension to the ligand by detecting fluoresce of the fluorescent dye/donor/acceptor on the surface as an indicator of the receptor binding to the ligand on the cell providing receptor-mediated tension and the receptor-mediated tension exerts forces exceeding a total force needed to denature the duplex.

In certain embodiments, methods further comprise identifying the receptor binding to the ligand by detecting fluoresce of the fluorescent dye/donor/acceptor on the cell as an indicator of the receptor binding to the ligand on the cell.

In certain embodiments, methods further comprise identifying the receptor binding to the ligand by detecting fluoresce of the fluorescent dye/donor/acceptor on the surface as an indicator of the receptor binding to the ligand on the cell.

In certain embodiments, the nucleobase polymer is DNA or RNA. In certain embodiments, the cell or vesicle is a platelet. In certain embodiments, ligand is an RGD peptide. In certain embodiments, the lipophilic agent is a sterol. In certain embodiments, lipophilic agent is a cholesterol.

In certain embodiments, methods further comprise a step of purifying or isolating an individual tagged cell reported herein, e.g., by flow cytometry. In certain embodiments, the tagged cell incorporated with the second strand having a lipophilic agent includes a unique identifying sequence/barcode, and methods include sequencing the unique identifying sequence/barcode.

### BRIEF DESCRIPTION OF THE SEVERAL VIEWS OF THE DRAWING(S)

FIG. 1 provides a scheme of tension-activated cell tagging (TaCT). TaCT is based on 3′ and 5′ end mechanical pulling of a DNA duplex that leads to dehybridization. The stability of a dsDNA 24 mer was evaluated as a function of applied force generated using oxDNA simulation with a loading rate of 2.81×10³ nm/s. The exponential moving average (EMA) indicates a 41 pN dehybridization transition. Flow cytometry histograms of NIH3T3 cells cultured on TaCT substrates as well as control surfaces with (−)cholesterol, (−)RGD, and (+)LatB were obtained.

FIG. 2A illustrates mapping integrin forces with a peeling probe. When integrin is in the inactive state, the peeling probe remains in the duplex form. As integrin binds and pulls on the RGD ligand, if F less than F peel, the peeling strand is intact, and if F greater than F peel, the duplex denatures and the Cy3B fluorescence turns on. FA proteins such as vinculin, talin, and FAK, as well as actin cytoskeleton participate intensively during the integrin force generation and mechanotransduction.

FIG. 2B shows a histogram of NIH3T3 cells showing the distribution of average % peel/μm² per cell after 1 h incubation on peeling probe substrate. Microscopy images showed that the phosphorylated FAK (pY397) colocalized with fixed integrin tension signal. MEF cells were incubated on peeling probe substrate for 40-45 min, fixed and stained with Rabbit anti-FAK pY397 and Alexa488 labeled secondary antibody, followed by imaging. Microscopy images showed that the actin stress fibers colocalized with fixed tension signal. MEF cells were incubated on peeling probe substrate for 60-90 min, fixed and stained with Alexa647-phalloidin, followed by imaging.

FIG. 3A schematically illustrates contemplated mechanisms of TaCT/peeling probe and TGTs. Peeling probe maps integrin tension greater than 41 pN as the BHQ2 strand is separated, and the RGD anchor remains despite the duplex dehybridization. In contrast, TGTs map integrin forces greater than 56 or 12 pN in the shearing or unzipping geometry. The force-induced rupture of the duplex generates a Cy3B turn-on fluorescence signal as the top BHQ2 strand separates from the Cy3B anchor strand. Unlike peeling strand, the loss of the RGD anchors in TGTs terminates mechanotransduction through integrins. Experiments indicated that the peeling probe does not perturbate mechanotransduction. Microcopy images were obtained of NIH3T3 cells incubated on peeling probe or TGT substrate for approximately 45-60 min. Microscopy images were obtained of NIH3T3 cells incubated on peeling probe or TGT substrates for approximately 90 min, fixed and stained for nucleus (DAPI), actin (SirActin), and YAP (Alexa488 conjugated antibody).

FIG. 3B shows data from quantitative analysis of the spreading area, % peel or % rupture, and YAP translocation for NIH3T3 cells incubated on three substrates for 60 min. For spreading area and tension quantification, data was collected from 3 biological replicates (n=227, 150, and 105 for cells on peeling probe, 56 pN TGT, and 12 pN TGT substrate). For analysis on YAP translocation, images in DAPI channel were used as masks to quantify the mean fluorescence intensity of nuclear YAP and cytoplasm YAP. Data was acquired from 3 biological replicates (total n=134, 98, and 86 cells for peeling probe, 56 pN TGT and 12 pN TGT substrate).

FIG. 4A illustrates Lat B inhibition of cells. Lat B early treatment inhibits integrin force generation by inhibiting actin polymerization. RICM and Cy3B microscopy images were obtained from MEF cells incubated on peeling probe substrate after early Lat B treatment. Cells were treated with 20 μM Lat B after 15 min of plating on the substrate and imaged after 45 min of incubation.

FIG. 4B illustrates that when integrin force signals are already generated on peeling probe substrate, if the force transmission is terminated by late Lat B treatment, with additional BHQ2 peeling strand, the peeling probe can rehybridize to the duplex form. RICM and raw Cy3B microscopy images were obtained of MEF cells treated with 20 μM Lat B 50 min after seeding. The tension signal remained after 10 min of Lat B treatment and diminished after the addition of excess BHQ2 peeling strand.

FIG. 4C shows data of quantitative analysis of tension signal changes after Lat B treatment and the addition of BHQ2 peeling strand in n=30 cells.

FIG. 5A illustrates a scheme for making flow cytometry measurements of the DNA strands uptake in solution. Mouse platelets were incubated with 50 nM of cholesterol peeling strand, TaCT duplex, load-bearing strand, TaCT duplex lacking RGD, and load-bearing strand lacking RGD in Tyrode's buffer for 30 min and spun down three times to wash away the excess oligos. The association was measured in both Atto647N and Cy3B channels by a flow cytometer.

FIG. 5B shows data for Cy3B and Atto647N median fluorescence intensity (MFI) of platelets incubated with different oligonucleotides.

FIG. 5C provides a scheme showing flow cytometry measurements of the concentration dependent incorporation of cholesterol peeling strand. MEF cells were incubated with 0, 1, 10, and 100 nM of cholesterol peeling strand for 1 h. The excess cholesterol peeling strand was washed away by spinning down in PBS three times, and the fluorescence intensity of cells was measured by a flow cytometer.

FIG. 5D shows data from representative histogram of cholesterol peeling strand association in cells. Linear relationship between cholesterol strand concentration and cell association was found, R2=0.977.

FIG. 5E provides a scheme showing the measurement of cholesterol peeling strand dissociation from the cell. NIH3T3 cells were incubated with 100 nM cholesterol peeling strand for 30 min and rinsed with PBS 3 times. Cells were divided into 6 aliquots and the remaining cholesterol strand in cells was measured every 30 min for 150 min by flow cytometry.

FIG. 5F shows a representative histogram and data from the decay of fluorescence in cells over time. The normalized MFI from 2 replicates was plotted to show the dissociation of cholesterol strand over time in NIH3T3 cells.

### DETAILED DISCUSSION

Before the present disclosure is described in greater detail, it is to be understood that this disclosure is not limited to embodiments described, and as such may, of course, vary. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps. The term “comprising” in reference to an oligonucleotide having a nucleic acid sequence refers to an oligonucleotide that may contain additional 5′ (5′ terminal end) or 3′ (3′ terminal end) nucleotides, i.e., the term is intended to include the oligonucleotide sequence within a larger nucleic acid. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein. The term “consisting of” in reference to an oligonucleotide having a nucleotide sequence refers an oligonucleotide having the exact number of nucleotides in the sequence and not more or having not more than a range of nucleotide expressly specified in the claim. For example, “5′ sequence consisting of” is limited only to the 5′ end, i.e., the 3′ end may contain additional nucleotides. Similarly, a “3′ sequence consisting of” is limited only to the 3′ end, and the 5′ end may contain additional nucleotides.

As used herein, the term “cell” refers to a biological compartment containing a lipid membrane and cytosol which may contain a nucleus containing genetic material, mitochondria, and other organelles. Thrombocytes (platelets) are unique cells derived from fragments of a progenitor megakaryocyte found in bone marrow. Platelets contain a lipid membrane, mitochondria, mRNA, proteins, granules but lack a nucleus. Platelets have a characteristic discoid shape typically about 1 to 3 μm in diameter.

A “label” refers to a detectable compound or composition that is conjugated directly or indirectly to another molecule to facilitate detection of that molecule. Specific, non-limiting examples of labels include fluorescent tags, enzymatic linkages, and radioactive isotopes. A label includes the incorporation of a radiolabeled compound or the covalent attachment of biotinyl moieties that can be detected by marked avidin (for example, streptavidin containing a fluorescent marker or enzymatic activity that can be detected by optical or colorimetric methods). Various methods of labeling molecules are known in the art and may be used. Examples of labels include, but are not limited to, the following: radioisotopes or radionucleotides (such as 18F, 35S or 131I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

As used herein, a “dye” refers to any entity that can fluoresce and/or quench fluorescence. In Fluorescence Resonance Energy Transfer (FRET), energy of an excited fluorophore (sometime referred to as a “donor”) is transferred to a neighboring acceptor molecule, e.g., limiting fluorescence of the donor fluorophore due to quenching (“quencher”). The distance between the donor and the quencher is related to the intensity of quenching. When donor fluorophore and quencher are separated, quenching is reduced or eliminated, and fluorescence is intensified. Likewise, when the donor and quencher are close, quenching is typically efficient, e.g., low intensity or dark. The range over which quenching occurs is unique for each fluorophore donor and quencher pair. The quencher molecule can be another fluorescent dye or a non-fluorescent dark quencher. See Johansson, Choosing Reporter-Quencher Pairs for Efficient Quenching Through Formation of Intramolecular Dimers, Methods in Molecular Biology, 2006, 335:17-29. Chapter 2 Fluorescent Energy Transfer Nucleic Acid Probes: Designs and Protocols.

Examples of fluorescent dyes include fluorescein dyes, cyanine dyes, fluorescein isothiocyanate (FITC) dyes, rhodamine-based dyes such as tetramethylrhodamine (TMR) dyes, carboxytetramethylrhodamine dyes (TAMRA), 6-(2-carboxyphenyl)-1,11-diethyl-3,4,8,9,10,11-hexahydro-2H-pyrano[3,2-g: 5,6-g′]diquinolin-1-ium dyes (ATTO™ dyes) and other such as 4,4-difluoro-4-bora-3a,4a-diaza-s-indacene (BODIPY) dyes, BODIPY FL™, Oregon Green 488™, Rhodamine Green™, Oregon Green 514™, TET™, Cal Gold™, BODIPY R6G™, Yakima Yellow™, JOE™, HEX™, Cal Orange™, BODIPY TMR-X™, Quasar-570/Cy3™, Rhodamine Red-X™, Redmond Red™, BODIPY 581/591™, ROX™, Cal Red/Texas Red™, BODIPY TR-X™, BODIPY 630/665-X™, Pulsar-650™, Quasar-670/Cy5™, Cy3.5™, and Cy5.5™. Examples of quenchers include fluorescein, rhodamine, and cyanine dyes, dabcyl, and Black Hole Quenchers™ (BHQs). Dark quenchers include dabcyl, QSY 35™, BHQ-0™, Eclipse™, BHQ-1™, QSY 7™, QSY 9™, BHQ-2™, ElleQuencher™, Iowa Black™, QSY 21™, BHQ-3™.

As used herein, the term “nucleic acid” is intended to mean a ribonucleic or deoxyribonucleic acid or analog thereof, including a nucleic acid analyte presented in any context; for example, a probe, target, or primer. A nucleic acid can include native or non-native bases. In this regard, a native deoxyribonucleic acid can have one or more bases selected from the group consisting of adenine, thymine, cytosine or guanine, and a ribonucleic acid can have one or more bases selected from the group consisting of uracil, adenine, cytosine, or guanine. It will be understood that a deoxyribonucleic acid used in the methods or compositions set forth herein can include uracil bases and a ribonucleic acid can include a thymine base. Exemplary non-native bases that can be included in a nucleic acid, whether having a native backbone or analog structure, include, without limitation, inosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine, 4-acetylcytidine, 2′-O-methylcytidine, 5-carboxymethylaminomethyl-2-thiouridine, 5-carboxymethylaminomethyluridine, dihydrouridine, 2′-O-methylpseudouridine, 2′-O-methylguanosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, N4-methylcytosine, 5-methylcytidine, N6-methyladenosine, 7-methylguanosine, 5-methylaminomethyluridine, 5-methylaminomethyl-2-thiouridine, beta-D-mannosylqueuosine, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl) carbamoyl) threonine, N-((9-beta-D-ribofuranosylpurine-6-yl)N-methyl-carbamoyl) threonine, uridine-5-oxoacetic acid-methylester, uridine-5-oxyacetic acid, wybutoxosine, pseudouridine, queuosine, 2-thiocytidine, 5-methyl-2-thiouridine, 2-thiouridine, 4-thiouridine, 5-methyluridine, N-((9-beta-D-ribofuranosylpurine-6-yl) carbamoyl) threonine, 2′-O-methyl-5-methyluridine, 2′-O-methyluridine, or 3-(3-amino-3-carboxypropyl) uridine.

Nucleic acids may have a backbone structure of 2′ deoxyribose 5′ monophosphate (the backbone moiety of a deoxyribonucleotide) or ribose 5′ monophosphate (the backbone moiety of a ribonucleotide); or an analogue of a 2′ deoxyribose 5′ monophosphate or ribose 5′ monophosphate, which when forming the backbone of a nucleic acid analogue, results in an arrangement of nucleobases that mimics the arrangement of nucleobases in nucleic acids containing a 2′ deoxyribose 5′ monophosphate or ribose 5′ monophosphate backbone, wherein the nucleic acid analogue is capable of base pairing with a complementary nucleic acid; examples of backbone moieties include amino acids as in peptide nucleic acids, glycol molecules as in glycol nucleic acids, threofuranosyl sugar molecules as in threose nucleic acids, morpholine rings and phosphorodiamidate groups as in morpholinos, and cyclohexenyl molecules as in cyclohexenyl nucleic acids.

A non-native base used in a nucleic acid can have universal base pairing activity, wherein it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine, which base-pairs with cytosine, adenine, or uracil. Alternatively, or additionally, oligonucleotides, nucleotides, or nucleosides including the above-described non-native bases can further include reversible blocking groups on the 2′, 3′ or 4′ hydroxyl of the sugar moiety.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleic acid may be single or double stranded or both, e.g., they may contain overhangs. Nucleobase polymers may contain naturally occurring or synthetically modified bases and backbones. In certain embodiments, a nucleobase polymer need not be entirely complementary, e.g., may contain one or more insertions, deletions, or be in a hairpin structure provided that there is sufficient selective binding. With regard to the nucleobases, it is contemplated that the term encompasses isobases, otherwise known as modified bases, e.g., are isoelectronic or have other substitutes configured to mimic naturally occurring hydrogen bonding base-pairs.

Nucleic acids may be chemically modified, e.g., within the sugar backbone or on the 5′ or 3′ ends. As such, in certain embodiments, nucleobase polymers disclosed herein may contain monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphon amidate, morpholin-2-ylmethanol, (2-(hydroxymethyl)morpholino) (piperazin-1-yl)phosphinate, or peptide nucleic acids or combinations thereof.

The terms “nucleic acid that hybridizes,” “nucleobase polymer that hybridizes,” or the like refers to a molecule capable of hybridizing to a single-stranded nucleic acid target. The nucleic acid or nucleobase polymer may be single stranded nucleic acid or analog containing a sufficiently small number of mismatches, additions, or deletions as long as the probe retains the ability to bind to the target.

In certain embodiments, a nucleobase polymer has a sequence of more than 7, 8, 9, 10, 11, 12, 13, 14, 15 or more nucleotides or nucleobases or continuous nucleotide nucleobases that is the reverse complement of a binding partner. In certain embodiments, the nucleobase polymer is less than 100, 50, or 25 nucleobases or base pairs. In certain embodiments, the nucleobase polymer is more than three nucleotides optionally less than seven, or more than four nucleotides optionally less than seven, or more than five nucleotides and optionally less than seven.

As used herein a “lipophilic agent” refers to a lipid or compound containing a lipid, i.e., a hydrophobic group that is highly insoluble in water. A lipid group is considered highly insoluble in water when the point of connection on the lipid to a compound is replaced with a hydrogen and the resulting compound has a solubility of less than 0.63×10⁻⁴% w/w (at 25° C.) in water, which is the percent solubility of octane in water by weight. See Solvent Recovery Handbook, 2nd Ed, Smallwood, 2002 by Blackwell Science, page 195. Examples of naturally occurring lipophilic agents include saturated or unsaturated hydrocarbon chains found in fatty acids, glycerolipids, cholesterol, steroids, polyketides, and derivatives. Non-naturally occurring lipids include derivatives of naturally occurring lipids, acrylic polymers, aromatic, and alkylated compounds and derivatives thereof.

The term “specific binding agent” refers to a molecule, such as a nucleic acid or proteinaceous molecule, that binds a target molecule with a greater affinity than other random molecules, nucleic acid, or proteins, e.g., at room temperature. Examples of specific binding agents include antibodies that bind an epitope of an antigen or a receptor which binds a ligand. “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of a random molecule, nucleic acid, or polypeptide.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000. In any of the embodiments disclosed herein the position of a ligand and a receptor may be switched.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, —(CH₂CH₂O)—, as —(OCH₂CH₂)— which may contain single, double, or triple bonds individually and independently between the R groups. If an R is branched with an Rₙ it may be terminated with a group such as —CH₃, —H, —CH—CH₂, —CCH, —OH, —SH, —NH₂, —N3, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, polyethylene glycol, alkoxyalkyl, and aromatic groups.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### Tension Activated Methods of Labeling and Purifying Cells

In certain embodiments, this disclosure relates to methods of fluorescently labeling a cell comprising contacting 1) a device comprising: a) a surface, b) a first strand of a nucleobase polymer linked to an area of the surface, wherein the first strand is conjugated to a ligand, and c) a second strand of a nucleobase polymer hybridized with the first strand forming a duplex, wherein the second strand is conjugated to a lipophilic agent, wherein the second strand is conjugated to a fluorescent dye; and 2) a cell comprising a receptor to the ligand and a lipid membrane applying receptor-mediated tension to the device whereby the lipophilic agent of the second strand incorporates to the lipid membrane of the cell thereby fluorescently labeling the cell.

In certain embodiments, this disclosure relates to methods of fluorescently labeling a cell comprising contacting 1) a device comprising: a) a surface, b) a first strand of a nucleobase polymer linked to an area of the surface, wherein the first strand is conjugated to a ligand, and c) a second strand of a nucleobase polymer hybridized with the first strand forming a duplex, wherein the second strand is conjugated to a lipophilic agent, wherein the second strand is conjugated to a fluorescent dye; and 2) a cell comprising a receptor to the ligand and a lipid membrane applying receptor-mediated tension to the device whereby the lipophilic agent of the second strand incorporates to the lipid membrane of the cell thereby fluorescently labeling the cell.

In certain embodiments, this disclosure relates to tagging cells or vesicles with agents using a tension activated device. In certain embodiments, a solid surface is conjugated to a double stranded nucleic acid complex wherein one strand is conjugated to the surface, and a second strand is conjugated to a lipophilic agent/lipid. In certain embodiments, the first and/or second strands are conjugated to a ligand and label, e.g., fluorescent dye. In certain embodiments, a cell or vesicle comprising a receptor that binds to the ligand provides tension on the double stranded complex to denature the strands peeling away the lipid conjugated strand resulting in cell membrane incorporating the lipid conjugated strand.

In certain embodiments, this disclosure relates to methods of fluorescently identifying a cell or vesicle for receptor ligand binding comprising contacting 1) a device comprising: a) a surface, b) a first strand of a nucleobase polymer linked to an area of the surface, wherein the first strand is conjugated to a ligand and c) a second strand of a nucleobase polymer hybridized with the first strand forming a duplex, wherein the second strand is conjugated to a lipophilic agent, wherein the second strand is conjugated to a fluorescent dye or other label; and 2) a cell or vesicle comprising a receptor to the ligand and a lipid membrane applying receptor-mediated tension to the device at the receptor opposite the ligand after the contacting step whereby the receptor binds the ligand providing receptor-mediated tension, e.g. due to stretching the duplex and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell providing a tagged cell or vesicle.

In certain embodiments, the label is a fluorescent dye. In certain embodiments, the first strand is conjugated to a fluorescent donor and the second strand is conjugated to a fluorescent quencher. In certain embodiments, the first strand is conjugated to a fluorescent quencher and the second strand is conjugated to a fluorescent donor.

In certain embodiments, methods further comprise identifying the receptor binding and/or quantifying the tension to the ligand by detecting the label or fluoresce of the fluorescent dye/donor/acceptor on the surface as an indicator of the receptor binding to the ligand on the cell providing receptor-mediated tension and the receptor-mediated tension exerts forces exceeding a total force needed to denature the duplex. In certain embodiments, the total force needed to denature the duplex is a threshold that exceeds 2, 3, 4, 5, 10, 50, or 100 piconewtons.

In certain embodiments, methods further comprise identifying the receptor binding to the ligand by detecting the label or fluoresce of the fluorescent dye/donor/acceptor on the cell as an indicator of the receptor binding to the ligand on the cell.

In certain embodiments, methods further comprise identifying the receptor binding to the ligand by detecting fluoresce of the label or fluorescent dye/donor/acceptor on the surface as an indicator of the receptor binding to the ligand on the cell.

In certain embodiments, the nucleobase polymer is DNA or RNA. In certain embodiments, the cell or vesicle is a platelet. In certain embodiments, ligand is an RGD peptide. In certain embodiments, the lipophilic agent is a sterol. In certain embodiments, lipophilic agent is a cholesterol.

In certain embodiments, methods further comprising a step of purifying or isolating an individual tagged cell reported herein, e.g., by flow cytometry. In certain embodiments, the tagged cell incorporated with the strand having a lipophilic agent includes a unique identifying sequence/barcode, and methods include sequencing the unique identifying sequence/barcode.

In certain embodiments, this disclosure relates to methods of barcoding a cell for identification of receptor ligand binding comprising contacting 1) a device comprising: a) a surface, b) a first strand of a nucleobase polymer linked to an area of the surface, wherein the first strand is conjugated to a ligand and c) a second strand of a nucleobase polymer hybridized with the first strand forming a duplex, wherein the second strand is conjugated to a lipophilic agent; and 2) a cell comprising a receptor to the ligand and a lipid membrane applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell providing a tagged cell.

In certain embodiments, with regard to any of the methods disclosed herein, the methods further comprise sequencing of the second strand of the tagged cell. In certain embodiments, the tagged cell is a single cell. In certain embodiments, the second strand contains a predetermined unique barcode sequence recorded on a computer readable medium.

In certain embodiments, this disclosure relates to methods of fluorescently identifying a cell for receptor ligand binding comprising contacting 1) a device comprising: a) a surface, b) a first strand of a nucleobase polymer linked to an area of the surface, wherein the first strand is conjugated to a ligand, and wherein the first strand is conjugated to a fluorescent donor, and c) a second strand of a nucleobase polymer hybridized with the first strand forming a duplex, wherein the second strand is conjugated to a lipophilic agent, wherein the second strand is conjugated to a fluorescent quencher, wherein the fluorescent quencher is located in sufficiently close proximity to the fluorescent moiety providing fluorescence quenching of the fluorescent donor; and 2) a cell comprising a receptor to the ligand and a lipid membrane applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell providing a fluorescently labeled cell and the fluorescent quencher is no longer located in proximity to the fluorescent donor to quench fluorescence. In certain embodiments, methods include the step of detecting and/or purifying the fluorescent cell.

In certain embodiments, methods include the step of detecting the label or fluorescent dye/donor/quencher as an indication that the ligand receptor binding or that the receptor applied a force on the duplex e.g., a force above a threshold value, indicative of the second strand denaturing or peeling away from the first strand and incorporating into the cell membrane.

In certain embodiments, this disclosure relates to methods of purifying a fluorescently tagged cell indicating receptor ligand binding comprising contacting 1) a device comprising: a) a surface, b) a first strand of a nucleobase polymer linked to an area of the surface, wherein the first strand is conjugated to a ligand, and wherein the first strand is conjugated to a fluorescent donor, and c) a second strand of a nucleobase polymer hybridized with the first strand forming a duplex, wherein the second strand is conjugated to a lipophilic agent, wherein the second strand is conjugated to a fluorescent quencher, and wherein the fluorescent quencher is located in sufficiently close proximity to the fluorescent donor providing fluorescence quenching; and 2) a cell comprising a receptor to the ligand and a lipid membrane applying a receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell providing a fluorescently tagged cell; and purifying the fluorescently tagged cell.

In certain embodiments, purifying a fluorescent cell is by separating fluorescent cells from non-fluorescent cells. In certain embodiments, purifying the cell is by flow cytometry or fluorescence activated cell sorting.

In certain embodiments, this disclosure relates to methods of fluorescently identifying a cell for receptor ligand binding comprising contacting 1) a device comprising: a) a surface, b) a first strand of a nucleobase polymer linked to an area of the surface, wherein the first strand is conjugated to a ligand, and wherein the first strand is conjugated to a fluorescent quencher, and c) a second strand of a nucleobase polymer hybridized with the first strand forming a duplex, wherein the second strand is conjugated to a lipophilic agent, wherein the second strand is conjugated to a fluorescent donor, and wherein the fluorescent quencher is located in sufficiently close proximity to the fluorescent donor providing fluorescence quenching; and 2) a cell comprising a receptor to the ligand and a lipid membrane applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell and the fluorescent quencher is no longer located in proximity to the fluorescent donor to quench fluorescence; and identifying the receptor binding to the ligand by detecting fluoresce of the fluorescent donor on the cell as an indicator of the receptor binding to the ligand on the cell.

In certain embodiments, this disclosure relates to methods of purifying a fluorescently tagged cell indicating receptor ligand binding comprising contacting 1) a device comprising: a) a surface, b) a first strand of a nucleobase polymer linked to an area of the surface, wherein the first strand is conjugated to a ligand, and wherein the first strand is conjugated to a fluorescent quencher, and c) a second strand of a nucleobase polymer hybridized with the first strand forming a duplex, wherein the second strand is conjugated to a lipophilic agent, wherein the second strand is conjugated to a fluorescent donor, and wherein the fluorescent quencher is located in sufficiently close proximity to the fluorescent donor providing fluorescence quenching; and 2) a cell comprising a receptor to the ligand and a lipid membrane applying a receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell and the fluorescent quencher is no longer located in proximity to the fluorescent donor to quench fluorescence providing a fluorescently tagged cell; and purifying the fluorescently tagged cell.

In certain embodiments, this disclosure relates to methods of fluorescently identifying a cell for receptor ligand binding comprising contacting 1) a device comprising: a) a surface, b) a first strand of a nucleobase polymer linked to an area of the surface, wherein the first strand is conjugated to a ligand, and c) a second strand of a nucleobase polymer hybridized with the first strand forming a duplex, wherein the second strand is conjugated to a lipophilic agent, wherein the second strand is conjugated to a fluorescent molecule; and 2) a cell comprising a receptor to the ligand and a lipid membrane applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell; and identifying the receptor binding to the ligand by detecting fluoresce of the fluorescent molecule on the cell as an indicator of the receptor binding to the ligand on the cell.

In certain embodiments, this disclosure relates to methods of purifying a fluorescently tagged cell indicating receptor ligand binding comprising contacting 1) a device comprising: a) a surface, b) a first strand of a nucleobase polymer linked to an area of the surface, wherein the first strand is conjugated to a ligand, and c) a second strand of a nucleobase polymer hybridized with the first strand forming a duplex, wherein the second strand is conjugated to a lipophilic agent, wherein the second strand is conjugated to a fluorescent molecule; and 2) a cell comprising a receptor to the ligand and a lipid membrane applying a receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell providing a fluorescently tagged cell; and purifying the fluorescently tagged cell.

In certain embodiments, this disclosure relates to methods of barcoding a cell for identification of receptor ligand binding comprising contacting 1) a device comprising: a) a surface, b) a first strand of a nucleobase polymer linked to an area of the surface, wherein the first strand is conjugated to a ligand and c) a second strand of a nucleobase polymer hybridized with the first strand forming a duplex, wherein the second strand is conjugated to a lipophilic agent and comprises unique identification sequence; 2) a cell comprising a receptor to the ligand and a lipid membrane applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the second strand no longer hybridizes to the first strand, the second strand becomes a single stranded nucleobase polymer in the area of the surface of the lipid membrane, whereby the lipophilic agent of the second strand incorporates to the lipid membrane of the cell providing a tagged cell.

In certain embodiments, for any of the methods disclosed herein, the method may further comprise sequencing of the second strand of the tagged cell. In certain embodiments, the tagged cell is a purified single cell prior to sequencing. In certain embodiments, the second strand contains a predetermined unique barcode sequence recorded on a computer readable medium.

### Molecular Mechanocytometry Using Tension-activated Cell Tagging (TaCT)

Flow Cytometry is Used to Measure Single-Cell Gene Expression by Staining Cells with fluorescent antibodies and nucleic acids. Tension-activated Cell Tagging (TaCT) was developed to fluorescently label cells based on the magnitude of molecular force transmitted through cell adhesion receptors. Fibroblasts and mouse platelets were analyzed after TaCT using conventional flow cytometry.

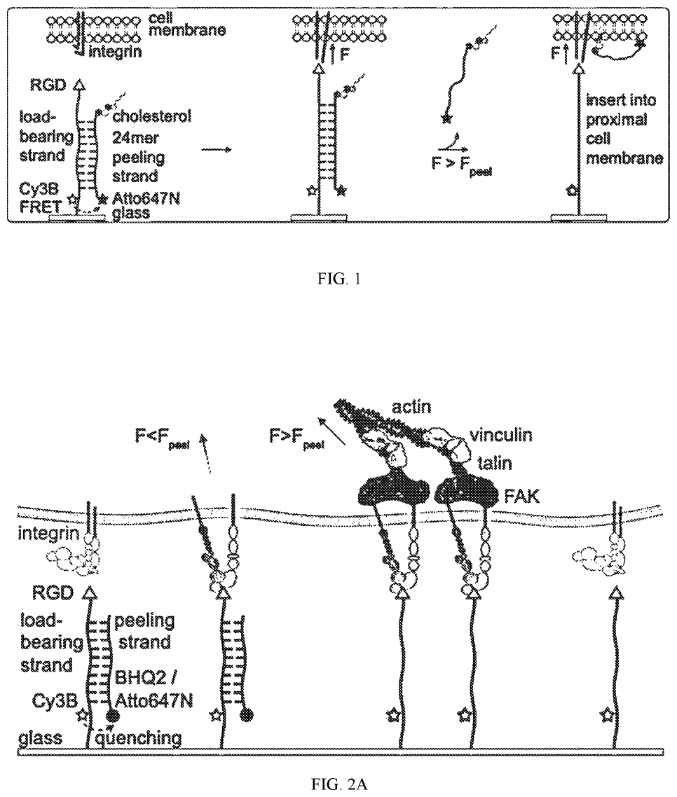

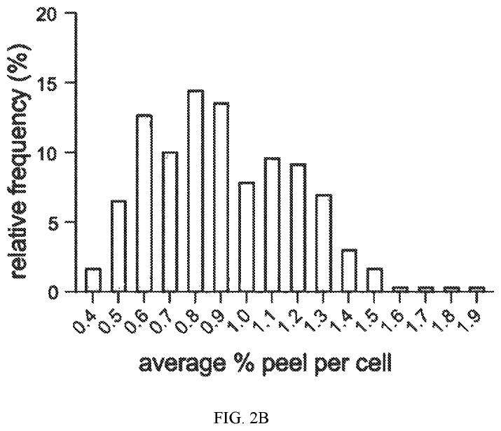

Tension-activated Cell Tagging (TaCT) enables flow cytometry based identification and sorting of mechanically active cells based on the molecular forces transmitted by their surface adhesion receptors. TaCT probes are engineered DNA duplexes that have a digital response to pN force and release a cholesterol-modified strand that spontaneously incorporates into the membrane of force-generating cells. TaCT takes advantage of the fundamental mechanism of double stranded DNA “peeling” under force. When a short DNA duplex is stretched from both ends of one of the strands (FIG. 1, 3′-5′ pulling), the duplex is destabilized, and denatures with sufficient force (peeling), leading to the complementary DNA separation.

In one example, TaCT probe is comprised of a load-bearing strand and a 24mer peeling strand. The load-bearing strand displayed the RGD integrin ligand at one terminus and was attached to the glass slide through its other terminus. The loading-bearing strand also incorporated an internal Cy3B dye. The complementary peeling strand was designed to release once force (F) is greater than F peel. The peeling strand termini were labeled with Atto647N and cholesterol. The TaCT probes were annealed and immobilized on streptavidin-coated glass slides. At rest, the probe is in the dsDNA form where Cy3B and Atto647N form a FRET pair. When F is greater than F peel, the Atto647N strand peels and dissociates, while the Cy3B dye on the load bearing strand is de-quenched. The FRET efficiency for TaCT probes was 93.8% and hence the Cy3B signal is enhanced by greater than 10-fold upon peeling. The probe density was measured at 5200 molecules/μm² using a quantitative fluorescence calibration, which allows one to convert the Cy3B fluorescence signal to a % peel.

To demonstrate that cell-generated forces drive DNA duplex peeling, mouse embryonic fibroblasts were plated on TaCT probe surfaces and then imaged using conventional fluorescence microscopy. A timelapse video showed cell spreading that coincided with Cy3B signal, confirming integrin binding and engagement with RGD ligands on the load-bearing strands. The Cy3B signal was primarily localized to the cell edge, consistent with the distribution of focal adhesions (FAs) and accumulated as a function of time. The growth of Cy3B signal corresponded to the loss of Atto647N signal and confirmed the force-induced peeling mechanism. This mechanism can generate high quality maps of integrins forces independent of cholesterol conjugation to the probe. Quantitative microscopy analysis of cells showed that 0.9% of probes peeled under each cell, equivalent to approximately 47 mechanical events/μm² with F greater than 41 pN. The observed tension signal colocalized with markers of FAs such as vinculin and phosphorylated focal adhesion kinase (FAK pY397), as well as actin stress fibers, confirming that forces were primarily transmitted by integrins within FAs. The peeling signal mirrors that of the turn-on tension gauge tether (TGT) probes but avoids termination of mechanotransduction which represents an advantage as a molecular force sensor. Control groups of cells treated with Latrunculin B (Lat B), which inhibits actin polymerization and disrupts force generation, showed significantly reduced peeling signal. Cells treated with Lat B 50 min after seeding did not show tension signal changes, confirming that the peeling mechanism is irreversible. Maps accumulated mechanical events over time. Interestingly, addition of soluble peeling strand led to the loss of the tension signal in cells treated with Lat B showing a simple approach to resetting tension signal and recording real-time events.

After confirming that DNA peeling consistently maps molecular traction forces, cholesterol-DNA cell tagging for TaCT was investigated. Cholesterol-DNA is contemplated to partition into the plasma membrane of cells. Cholesterol-single stranded DNA conjugates were approximately 50-fold more effective at tagging cells compared to cholesterol-double stranded DNA conjugates. Cholesterol-single stranded DNA membrane-association was linearly proportional to the soluble conjugate concentrations tested. The stability of TaCT tags was examined by incubating cholesterol-single stranded DNA with cells, washing, and measuring the loss of DNA as a function of time. Approximately 20-30% of the cholesterol-tethered DNA dissociated at 90 min. Accordingly, the cells were incubated on the surface for 1 h, which allowed for focal adhesion formation and TaCT method to proceed while limiting cholesterol dissociation.

NIH3T3 cells were seeded onto the TaCT substrates, allowed to spread, and collected by gentle scraping. The TaCT signal was then measured by flow cytometry. Probes lacking RGD, probes lacking cholesterol, as well as full TaCT probes cells treated with Lat B were used as negative controls. The resulting flow cytometry histograms showed a heterogeneous distribution of TaCT signal matching the tension signal distributions observed under microscopy. The cells on the TaCT substrate showed 27.6% force positive population compared to 0.6%, 1.1%, and 3.5% in (−)cholesterol, (−)RGD, and (+) Lat B controls, respectively. The Atto647N geometric mean fluorescence intensity (gMFI) of cells incubated on TaCT showed a greater than 2-fold increase compared to controls, whereas the Cy3B gMFI did not change. Taken together, these results confirmed that TaCT is triggered by force transmission through the integrin-RGD complex, followed by cholesterol-mediated membrane incorporation. Background tagging of cells due to cell spreading and proximity to the surface leads to negligible TaCT signal.

Experiments were performed to determine whether this strategy works in primary cells. Platelets were chosen for this demonstration as mechanical forces play a crucial role in platelet activation, aggregation, and clot retraction, which are necessary steps in coagulation. When mouse platelets were seeded onto the TaCT surface for 30 min, a loss of Atto647N signal was noted that was coupled with an increase in Cy3B. This confirmed that platelet integrin force transmission was sufficient to mediate DNA peeling with F greater than 41 pN. The Atto647N depletion signal did not colocalize exclusively with the Cy3B turn-on signal, which may be due to the accumulation of cholesterol-DNA probes at the basal face of the cell membrane. Nonetheless, the TaCT signal was measured by flow cytometry, which showed 13.3% force positive population. Control experiments withholding the divalent cations Mg2+ or Ca2+ necessary for full integrin activation showed minimal TaCT signal and force positive population.

To showcase that TaCT is an effective method to report cell receptor forces, a Rho-associated protein kinase (ROCK) inhibitor, Y27632, was used to disrupt the force transmission through cytoskeleton and measure the dose-dependent response in MEF cells. Y27632 inhibits ROCK kinases by competing with ATP binding, and further result in decreased myosin activity, destabilization of actin filament and abolished stress formation. With Y27632 pre-treatment, MEF cells showed decreased Cy3B tension signal. TaCT signal measured by flow cytometry showed a clear dose-dependent curve with an IC₅₀ of 773 nM, agreeing well with microscopy measurements and the literature reported IC₅₀ values that range from hundreds of nM to low μM.

After demonstrating TaCT with a single population of cells, experiments were performed to determine whether one can analysis a binary mixture of cells with differing mechanical activity. Parental MEF cells (wildtype, WT) and MEF cells with vinculin null (vin−) were used for this demonstration. Vinculin is an adaptor protein that localizes to FAs linking the cytoskeleton to adhesion receptors and aids in FA maturation. TaCT was performed on these two cell types separately. The vin− cells were stained with cell tracker dye, CMFDA, before plating onto TaCT substrates. To ensure that the TaCT signal is not due to differential uptake, cholesterol uptake of WT and vin− cells was measured by flow cytometry in parallel with each TaCT assay. WT cells had larger spreading area and produced more tension signal compared to vin− cells in the Cy3B channel. Flow cytometry also showed that there was more Atto647N signal in WT compared to vin−. Experiments were performed to determine whether this differential tension signal could be detected when the WT and vin− were co-cultured. The WT cells displayed more TaCT tension signal by microscopy compared to vin− cells despite effectively spreading on the substrate. The WT TaCT signal decreased slightly when these cells were co-cultured, with a 24.4% force positive population compared to 5.2% for vin− in the mixed population. The minimal TaCT signal change observed for vin− cells in co-culture indicated that TaCT tags remain associated with target cells within the experimental time window. Together, these results confirm that TaCT can distinguish mechanically active subpopulations in heterogeneous mixtures of cells.

The 3′-5′ mediated DNA peeling was used in DNA tension probes to map the molecular forces generated by cells and to enable high-throughput flow-cytometry based detection of mechanically active cells. TaCT is orthogonal to biochemical analysis using antibodies and nucleic acids. Hence, the technique allows for one to link single-cell mechanical properties with protein and nucleic acid expression levels.

TABLE 1
nucleic acids used in the experiments

| construct | oligo | 5′ |
|---|---|---|
| TaCT/peeling | load-bearing strand | /5ThioMC6-D/ |
| probe | 24 mer 5′NH2 peeling strand | /5AmMC6/ |
| | 24 mer Atto647N and | /5ATTO647NN/ |
| | cholesterol peeling strand | |
| | 24 mer BHQ2 peeling strand | /5BHQ_2/ |
| control duplex | non-fluorescent load-bearing | /5ThioMC6-D/ |
| | strand | |
| | non-fluorescent peeling strand | /5AmMC6/ |
| TGTs | TGT 12 pN bottom strand | /5AmMC6/ |
| | TGT 56 pN bottom strand | /5Biosg/ |
| | TGT top quencher strand | /5Hexynyl/ |

| sequence (5′ to 3′) | | 3′ |
|---|---|---|
| TTT TTT TTT TAG TGA GCT | SEQ ID NO: 1 | /3Bio/ |
| CTG AAG TCT TAG AAC T/ | | |
| iAmMC6T/T TT | | |
| AG TTC TAA GAC TTC AGA | SEQ ID NO: 2 | — |
| GCT CAC T | | |
| AG TTC TAA GAC TTC AGA | SEQ ID NO: 2 | /3cholTEG/ |
| GCT CAC T | | |
| AG TTC TAA GAC TTC AGA | SEQ ID NO: 2 | — |
| GCT CAC T | | |
| TTT TTT TTT TCA TAC GGT | SEQ ID NO: 3 | /3Bio/ |
| TAT AGA GTA G/ | | |
| iAmMC6T/T TT | | |
| CTA CTC TAT AAC CGT ATG | SEQ ID NO: 4 | — |
| CGC ATC TGT GCG GTA TTT | SEQ ID NO: 5 | /3Bio/ |
| CAC TTT | | |
| TT T/iUniAmM/C GCA TCT | SEQ ID NO: 6 | |
| GTG CGG TAT TTC AC | | |
| GTG AAA TAC CGC ACA GAT | SEQ ID NO: 7 | /3BHQ-2/ |
| GCG | | |

### Peeling Probe Offers Significant Advantages as a Tension Sensor Compared to TGTs.

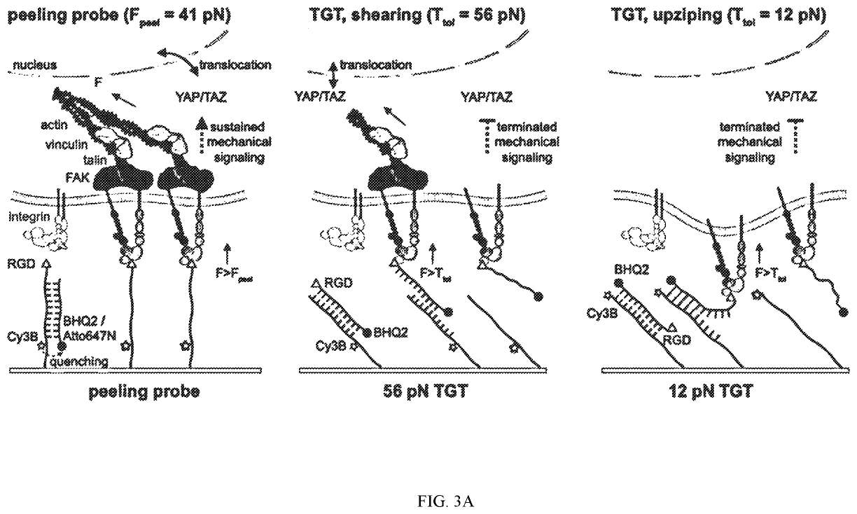

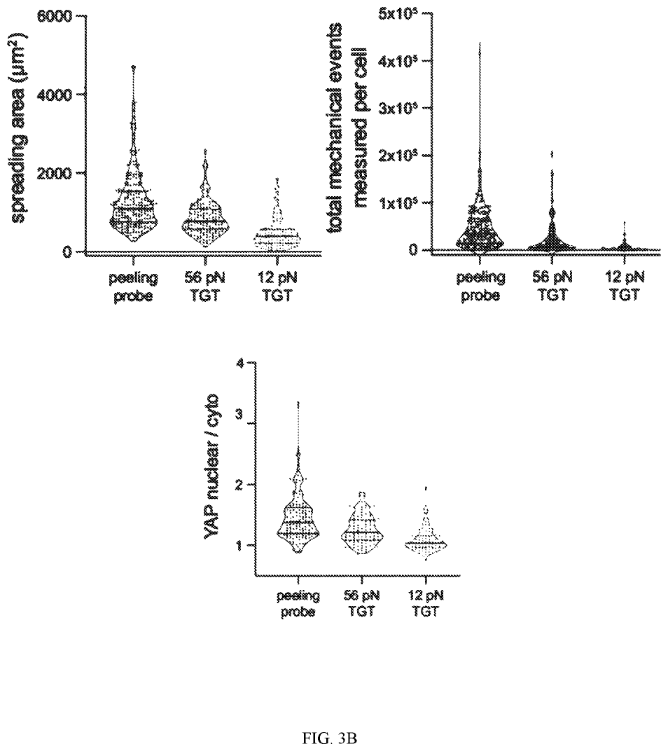

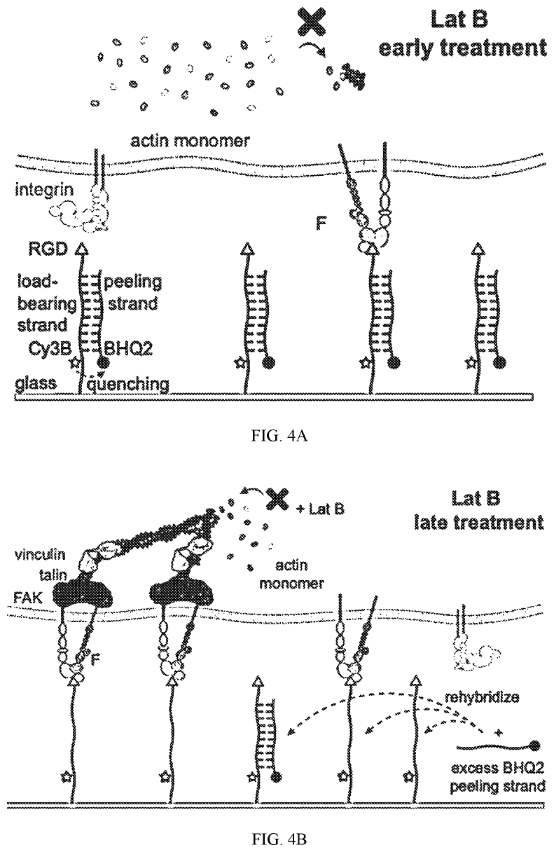

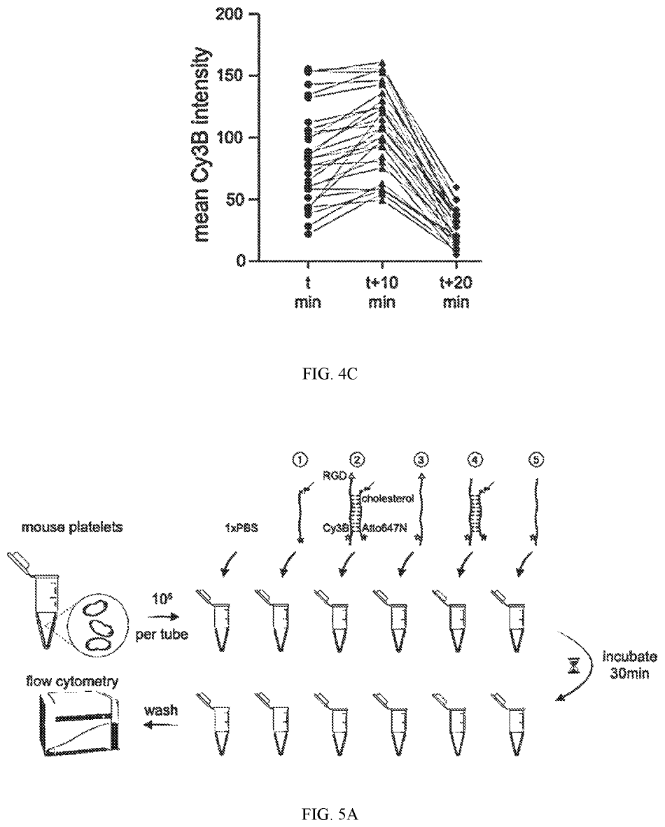

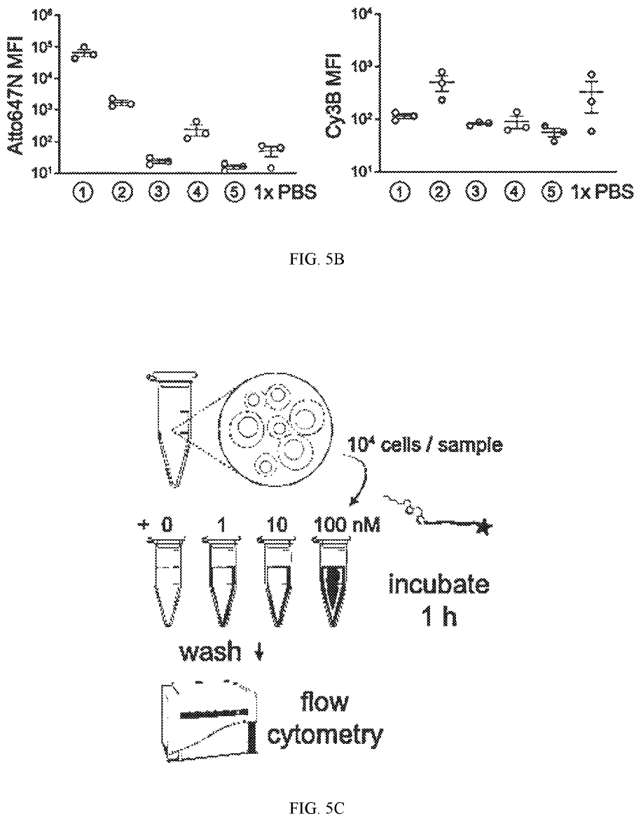

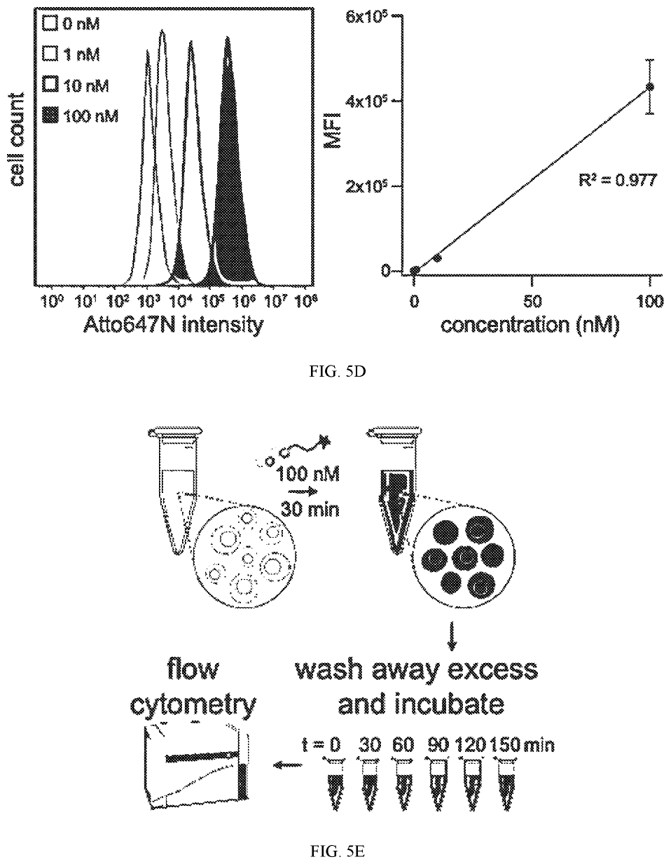

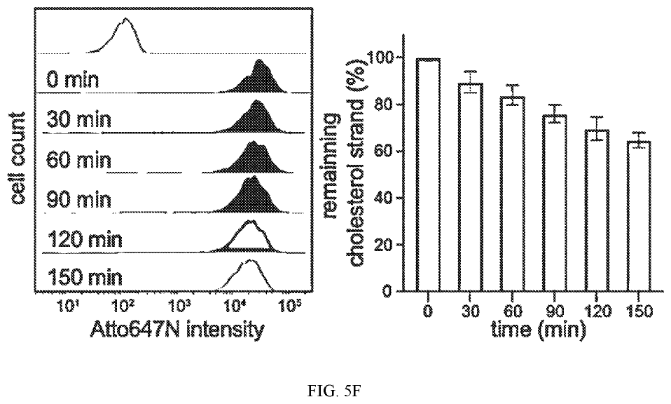

Wang et al. report a tension gauge tether (TGT) approach in which where a ligand is immobilized to a surface through a rupturable tether before receptor engagement. Science, 40 (6135): 991-4. Unlike TGTs, the force-induced denaturation of the peeling probe does not result in termination of the mechanical force experienced by adhesion receptors (FIG. 3A). Cell spreading area, integrin tension maps were compared, and the mechanical signaling outcomes for cells incubated on peeling probe or TGT substrates. Quantitative analysis (FIG. 3B) showed that the spreading area for cells incubated on the peeling probe substrate was significantly larger than that of cells incubated on 12 and 56 pN TGT substrates. The total number of mechanical events was calculated by multiplying the probe density, cell area, and the % peel or % rupture of cells incubated on the three different substrates. There were significantly more total mechanical pulling events for cells incubated on peeling probe substrate versus TGTs, possibly due to two reasons: first, the F peel is approximately 41 pN, which is less than the T tol for 56 pN TGT; and second, loss of integrin-ligands for TGT substrates modulates cell signaling. Based on the differences in the cell spreading area and tension profile, experiments were performed to further analyse mechanical signaling by quantifying Yes-associated protein (YAP) translocation to the nucleus. YAP translocation to the nucleus is regulated by focal adhesion signaling, F actin organization, and mechanotransduction. YAP nuclear signaling is important in regulating cell morphology, proliferation, and plasticity. YAP staining showed that cells incubated on peeling probe substrates had the highest YAP nuclear/cyto ratio, followed by 56 pN and then 12 pN TGTs. This result confirms that terminating mechanotransduction through integrin dampens YAP translocation into the cell nucleus, which impairs transcription activation. This result suggests that the peeling probe is better suited to decouple tension sensing from receptor force manipulation within cells in mechanobiology studies, which is advantageous.

1. A method of fluorescently labeling a cell comprising contacting
1) a device comprising:
a) a surface,
b) a first strand of a nucleic acid linked to an area of the surface, wherein the first strand is conjugated to a ligand, and
c) a second strand of a nucleic acid hybridized with the first strand forming a duplex, wherein the second strand is conjugated to a lipophilic agent, wherein the second strand is conjugated to a fluorescent dye; and
2) a cell comprising a receptor to the ligand and a lipid membrane applying receptor-mediated tension to the device whereby the lipophilic agent of the second strand associates with the cell membrane thereby fluorescently labeling the cell.

2. The method of claim 1, wherein the first strand is conjugated to a fluorescent dye, and the fluorescent dye is a fluorescent donor, and the second strand fluorescent dye is a fluorescent quencher.

3. The method of claim 2 further comprising detecting fluoresce of the fluorescent donor on the surface.

4. The method of claim 1, wherein the first strand is conjugated to a fluorescent dye, and the second strand is conjugated to a fluorescent dye.

5. The method of claim 4, further comprising identifying the receptor binding to the ligand by detecting fluorescence of the fluorescent dye conjugated the second strand on the cell and detecting the fluorescence of the fluorescent dye conjugated the first strand on the surface.

6. The method of claim 1 wherein applying receptor-mediated tension exceeds 10 piconewtons.

7. The method of claim 1 further comprising purifying the fluorescently labeled cell by fluorescent activated cell sorting.

8. The method of claim 1, wherein the nucleobase polymer is DNA or RNA.

9. The method of claim 1, wherein the cell is a platelet.

10. The method of claim 1, wherein the ligand is an RGD peptide.

11. The method of claim 1, wherein the lipophilic agent is a sterol.

12. The method of claim 1, wherein the lipophilic agent is a cholesterol.
